(19) United States
(12) Patent Application Publication
(10) Pub. No.: US 2011/0313403 A1
(43) Pub. Date: Dec. 22, 2011

(54) LOCKING MECHANISM FOR A CATHETER

(76) Inventors: Amy Lee Hruska, Indianapolis, IN (US); Geoffrey P. Grant, Bloomington, IN (US); David R. Lessard, Bloomington, IN (US); Steven D. Anderson, Bloomington, IN (US); Michael W. Hardert, Bloomington, IN (US)

(21) Appl. No.: 13/129,906

(22) PCT Filed: Nov. 12, 2009
(86) PCT No.: PCT/US09/64102

§ 371 Date: Sep. 7, 2011

Related U.S. Application Data
(60) Provisional application No. 61/118,043, filed on Nov. 26, 2008.

(51) Int. Cl. A61M 27/00

(52) U.S. Cl. 604/540

(57) ABSTRACT

A locking mechanism (15) for a catheter includes a main body (20), a tip (30), and a locking arm (50). The locking arm includes a pivotable portion (56) selectively receivable in a contoured outer surface portion of the main body (24). One end of a tubular member is engageable with the distal end of the tip, and the other end is insertable into a body cavity, e.g., for drainage of fluid therefrom. A tension member (14), e.g. suture, extends through the locking mechanism and the tubular member, and is actuable to maneuver the inserted end of the tubular member into a desired configuration, such as a loop or a pigtail (12). An end of the tension member is receivable along the main body contoured surface following actuation, and is lockable therein upon receipt of the locking arm pivotable portion in the contoured surface, thereby locking the tubular member end in the desired configuration.

### BACKGROUND

1. Technical Field

This invention relates to a locking mechanism for use with a medical device, such as a catheter. More particularly, the invention relates to a locking mechanism for use with a drainage catheter, for drawing and maintaining the distal end of the catheter into a desired configuration to anchor the catheter in an internal body cavity of a patient.

2. Background Information

Percutaneously inserted drainage catheters are now in widespread use in the medical field. For example, such catheters are often used in suprapubic catheterization of the bladder in order to drain the bladder following surgery, or when the genitourinary system is plugged by an obstruction. Other percutaneously inserted catheters are commonly used to drain the kidney or biliary system, as well as to drain abscesses, other sites of fluid collection, and other viscera. Still other percutaneously inserted catheters are useful for other purposes, such as gastrostomy feeding tubes.

These catheters are typically introduced into the patient by means of a large hypodermic needle or trocar, which pierces the abdominal wall. A wire guide is inserted through the needle and then removed. The catheter tube with a stiffener positioned therein is then passed over the wire guide into the cavity. The stiffener and wire guide are withdrawn, leaving the catheter in the desired cavity. With respect to the bladder, the advantage of this technique is that irrigation and infection of the urinary tract is minimized. However, a problem that may be encountered with sue of such catheters is that they are subject to dislodgement or removal by movement of the body or, for example, by the emptying of the bladder.

Various catheters have been developed with so-called pigtail loops at their distal ends for ensuring drainage of the cavity and preventing accidental removal therefrom. The pigtail loop is tightened by pulling on the proximal end of a flexible tension member, which extends through the catheter. The proximal end of this tension member is held in place by any one of a number of retention means. In one case, the proximal end of the flexible tension member is held in place by axially placing a hollow cap into or over the proximal end of the catheter tube, thus trapping the flexible tension member of which the protruding end may then be cut. A problem with this catheter design is that once the protruding end of the flexible tension member is cut, the hollow cap may slip or be inadvertently removed. As a result, the shortened flexible tension member moves distally, releases the pigtail, and is either difficult or impossible to retrieve.

In another case, the flexible tension member is trapped between two or more hollow tubes, one of which is slidably inserted axially into the other. A short length of the flexible member is generally left hanging from the catheter tube so that if the flexible tension member becomes loose, it may be retightened. Alternatively, an external sleeve is slid over the flexible tension member protruding from the side of the catheter tube of which the flexible tension member is then wound around and tied about the sleeve. Although well-suited for its intended purpose, this catheter design leaves the flexible tension member exposed at the proximal end of the catheter. As a result, a physician must grasp and pull on the flexible tension member to secure or tie it about the proximal catheter end. In addition, a patient can untie the exposed flexible tension member and require the assistance of hospital personnel to retie the member. Furthermore, when the flexible tension member is inadvertently released, the retaining loop at the distal end is released with the possibility of the catheter being withdrawn from the patient.

In yet another case, the proximal end of a flexible tension member is bent and secured between two cooperating locking members, one of which is laterally movable across the path of the flexible tension member. A problem with this catheter design is that the locking members bend or deform the flexible tension member. As a result, the locking members may break or cut the flexible tension member, thereby inadvertently releasing the distally positioned loop and allowing the withdrawal of the catheter from the patient.

Another such device is described in U.S. Pat. No. 5,399,165, incorporated by reference herein. The '165 patent describes a drainage catheter with a distal member end that is lockable in a desired loop configuration. The lockable drainage catheter includes a flexible tension member that extends through a lockable connector positioned about the proximal member end, for drawing the distal member end into a loop. The lockable connector includes a resilient material sleeve with a sleeve passage extending longitudinally therethrough for positioning the tension member therein. The sleeve is positioned in a passage of the connector adjacent a channel, wherein a pivotally attached lever is positioned. When the lever is pivoted toward the connector into a fixed position, a cam surface of the lever compresses the sleeve and locks thereabout to maintain the loop formed in the distal member end. Although the drainage catheter and locking mechanism described in the '165 patent are generally effective for their intended purpose, the lockable connector portion of the device is somewhat bulky and includes some sharp corners, both of which may make the device uncomfortable for some patients. In addition, protection against leakage is generally not provided until the lever is manipulated into the closed position.

It is desired to provide a locking mechanism for use with a catheter, such as a drainage catheter, that avoids the problems encountered with prior art catheters and locking mechanisms. It is further desired to provide a locking mechanism for a catheter that is effective for maintaining a desired configuration of the distal portion of the catheter when positioned in a body cavity.

### BRIEF DESCRIPTION OF THE DRAWINGS

FIG. 1 is a perspective view of a catheter with a locking mechanism according to an embodiment of the present invention, partially broken away, showing the locking mechanism in the locked position with the distal end of the catheter locked into a loop configuration;

FIG. 1A is an exploded view of the catheter of FIG. 1, in the unlocked position;

FIG. 2 is a sectional view of the locking mechanism of the catheter shown in FIG. 1;

FIG. 3 is a sectional view of the catheter of FIG. 1, showing the locking mechanism in an unlocked position and the distal end of the catheter in a straightened configuration;

FIG. 4 is a side sectional view of the main body portion of the locking mechanism;

FIG. 5 is a proximal end view of the main body portion shown in FIG. 4;

FIG. 6 is a distal end view of the main body portion shown in FIG. 4;

FIG. 7 is a side sectional view of the tip portion of the locking mechanism, also illustrating a tubular member extending distally from the tip portion;

FIG. 8 is a proximal end view of the tip portion shown in FIG. 7;

FIG. 9 is a distal end view of the tip portion shown in FIG. 7;

FIG. 10 is a proximal end view of the locking arm portion of the locking mechanism;

FIG. 11 is a distal end view of the locking arm portion shown in FIG. 10;

FIG. 12 is a side sectional view of the locking arm portion shown in FIG. 10; and

FIG. 13 is an enlarged side view of an elastomeric plug that is positioned in an aperture in the locking arm.

### DETAILED DESCRIPTION OF THE DRAWINGS AND THE PRESENTLY PREFERRED EMBODIMENTS

For the purposes of promoting an understanding of the principles of the invention, reference will now be made to the embodiments illustrated in the drawings, and specific language will be used to describe the same. It should nevertheless be understood that no limitation of the scope of the invention is thereby intended, such alterations and further modifications in the illustrated apparatus, and such further applications of the principles of the invention as illustrated therein being contemplated as would normally occur to one skilled in the art to which the invention relates.

In the following discussion, the terms “proximal” and “distal” will be used to describe the opposing axial ends of the catheter, as well the opposing axial ends of component features, such as the locking mechanism. The term “proximal” is used in its conventional sense to refer to the end of the catheter, or component feature, that is closest to the operator during use. The term “distal” is used in its conventional sense to refer to the end of the catheter, or component feature, that is initially inserted into the patient, or that is closest to the patient during use.

In one form, the present invention is directed to a catheter, such as a drainage catheter, having a locking mechanism disposed at the proximal end of the catheter. The catheter has a tubular member, the distal end of which is lockable into a desired configuration, such as a loop or a pigtail, for anchoring the catheter in a body cavity, such as the bladder. A flexible tension member, such as a suture, extends through the locking mechanism and to the distal end of the tubular member for use in drawing the distal end into the desired configuration, whereupon the locking mechanism is activated to maintain the distal end in this configuration.

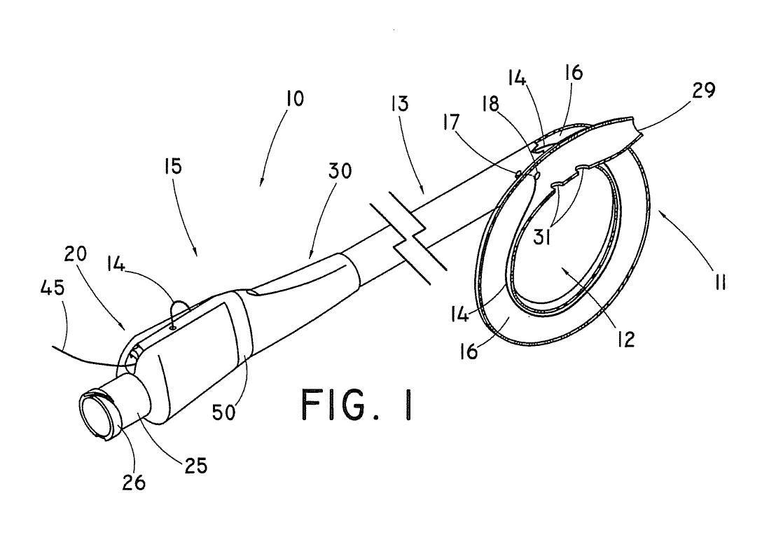

FIG. 1 is a perspective view, partially broken away, of one embodiment of a catheter 10 according to the present invention. In this figure, catheter 10 is a drainage catheter, and the catheter is shown in the locked position. FIG. 1A is an exploded view of the drainage catheter of FIG. 1, in the unlocked position. As illustrated, drainage catheter 10 includes a locking mechanism 15 positioned at the proximal end of the catheter, and an elongated tubular member 13 extending in the distal direction from the locking mechanism. Preferably, tubular member 13 tapers to an open distal tip 29.

In the view of catheter 10 illustrated in FIG. 1, the distal end 11 of tubular member 13 has been maneuvered into the desired configuration, in this case a loop 12. Placing the distal end of the tubular member into the desired configuration, e.g., a loop or a pigtail, inhibits unintended withdrawal or displacement of the catheter from its position in the designated body cavity, e.g. the bladder of a patient. As best shown in FIG. 1A, locking mechanism 15 comprises a main body portion 20, a tip portion 30, and a locking arm 50.

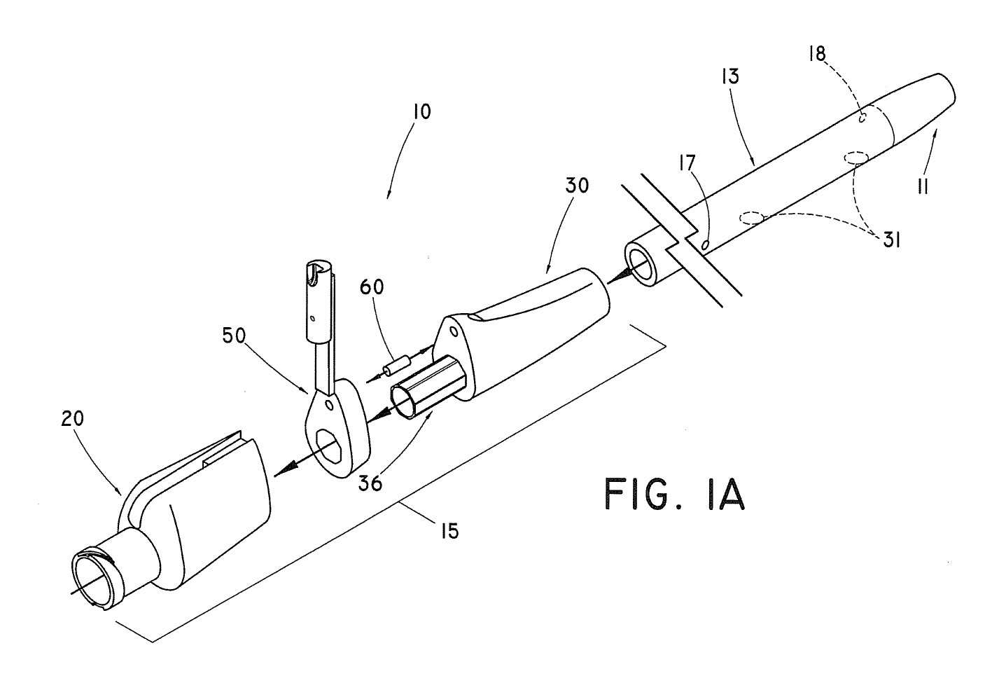

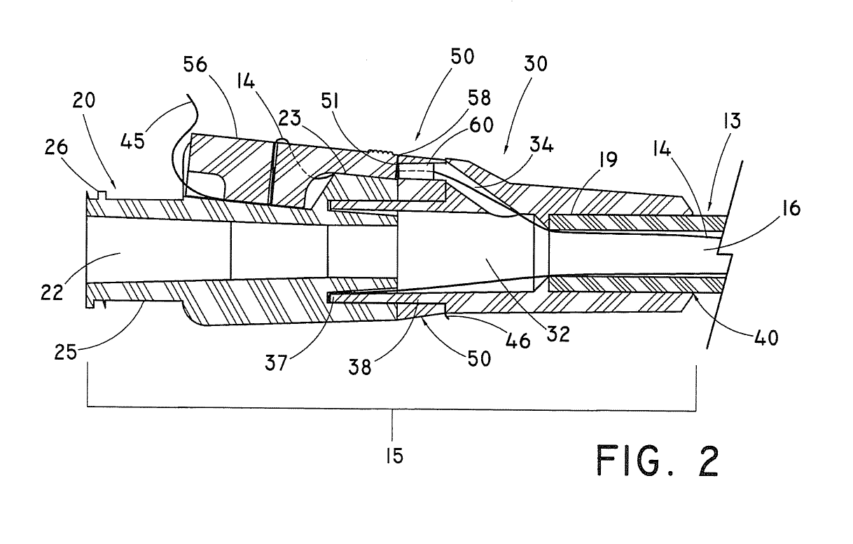

FIG. 2 is a sectional view of the locking mechanism 15 of FIG. 1, also showing the locking mechanism in the locked position. FIG. 3 is a sectional view of the drainage catheter, showing locking mechanism 15 in an unlocked position. In the unlocked position depicted in FIG. 3, the distal end of the catheter is in a straightened configuration as shown.

As shown in the figures, tubular member 13 has a passageway 16 extending longitudinally between distal member end 11 and the locking mechanism 15. Tubular member 13 preferably includes one or more drainage ports 31 communicating with passageway 16. When present, drainage ports 31 are positioned proximate tubular member distal end 11 for receiving fluid to be drained from the body cavity. Ports 31 may have any geometrical configuration, such as the circular ports shown in the figures, suitable for permitting drainage of fluid into the interior of the tubular member. Tubular member 13 further includes ports 17, 18 positioned proximate distal member end 11 and communicating with passageway 16 (FIG. 3). Ports 17 and 18 are spaced apart a predetermined length, which length corresponds to, e.g., the circumference of the desired loop configuration. Tubular member 13 also preferably includes an open distal tip 29 suitable for receiving fluid for drainage from the cavity. Those skilled in the art will appreciate that the number, shape, size, and arrangement of ports at the distal end of the tubular member is generally not critical, and may be varied for a particular case as long as sufficient fluid from the body cavity may be received for drainage through the ports and/or distal tip 29.

A tension member 14, such as a suture, extends along the tubular member passageway and through ports 17 and 18. Tension member 14 is arranged such that a first end 45 of the tension member extends outwardly from locking mechanism 15 to enable grasping and drawing of the tension member end to form the desired loop configuration at the distal portion 11 of the tubular member 13. Tension member 14 further includes second end 46. Preferably, second end 46 is fixedly captured between main body portion 20 and tip portion 30 during assembly of the device, as shown in FIGS. 2 and 3. Any portion of second end 46 extending outside of the locking mechanism may be trimmed away.

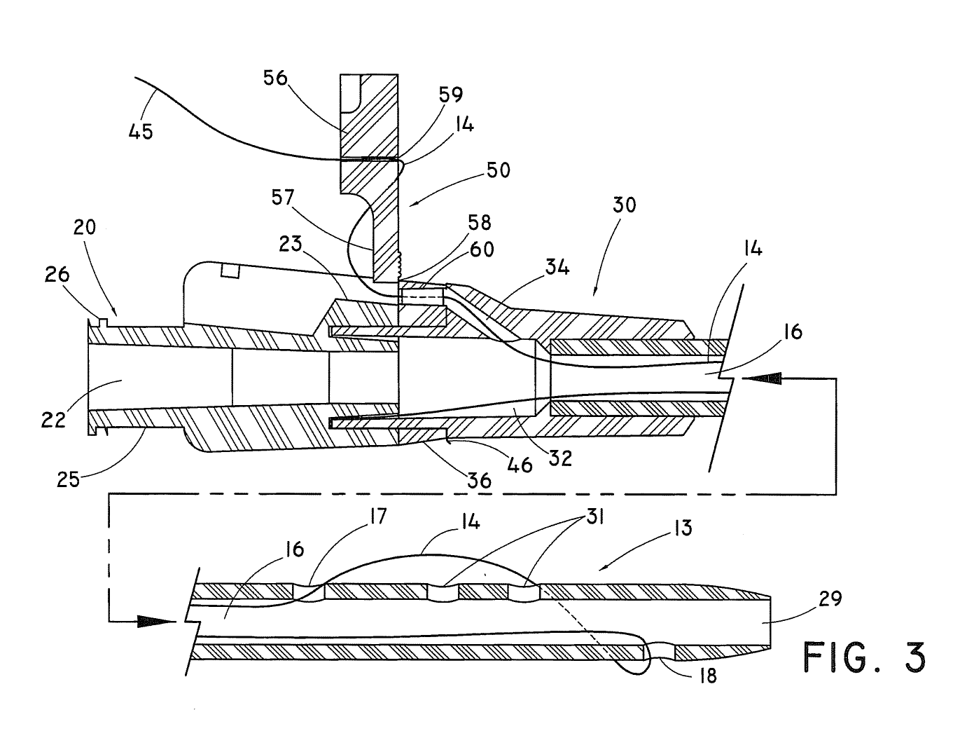

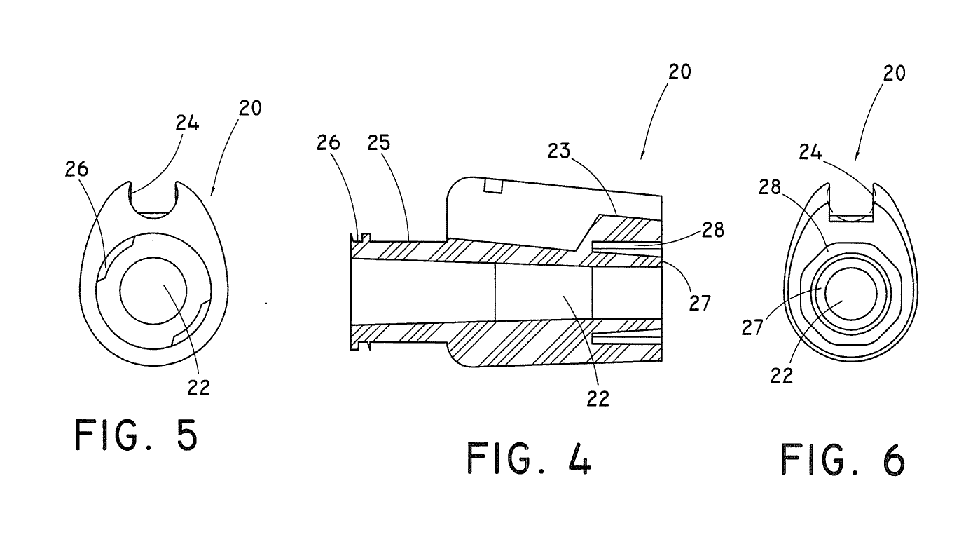

Main body portion 20 of the locking mechanism is further illustrated in FIGS. 4-6. FIG. 4 is a side sectional view of main body portion 20. FIG. 5 is an end view from the proximal end of main body portion 20, and FIG. 6 is an end view from the distal end of the main body portion. Main body portion 20 includes a longitudinal passageway 22 extending therethrough. Main body portion 20 also includes a contoured portion, such as cradle portion 24, for receiving pivotable member 56 of locking arm 50, as described herein. Cradle portion 24 may be shaped, such as by the inclusion of ledge 23, for receiving a complementary-shaped feature of the locking arm pivotable member 56.

In the embodiment shown, the proximal end of main body portion 20 includes an extension member 25 that leads to a connector 26, such as a Luer connector. Connector 26 is shaped for connecting main body portion 20 to another device, such as a fluid collection system (not shown), by well-known means. Main body portion 20 may also include an interior distal ring 27 defining a channel 28. Channel 28 extends along the distal circumference of the body portion. Channel 28 is sized and shaped for receiving a complementary-shaped ring portion 37 of tip 30, when locking mechanism 15 is fully assembled.

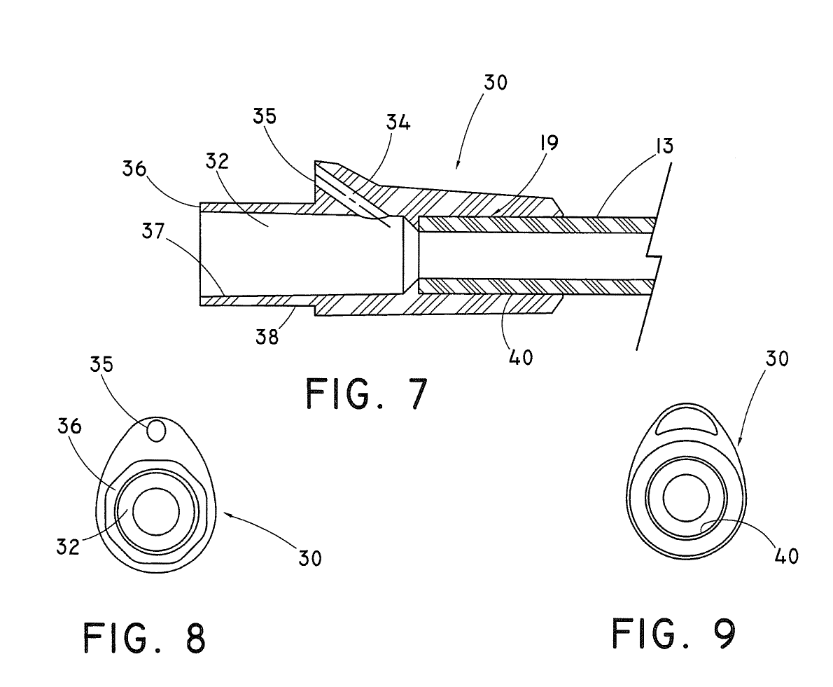

Tip portion 30 is further illustrated in FIG. 7-9. FIG. 7 is a side sectional view of tip portion 30. The view of FIG. 7 also illustrates tubular member 13 extending distally from tip portion 30. FIG. 8 is a proximal end view of the tip portion shown in FIG. 7, and FIG. 9 is a distal end view of the tip portion shown in FIG. 7. Tip portion 30 includes a longitudinal first passageway 32 extending therethrough. When the locking mechanism is fully assembled, tip portion first passageway 32 communicates with main body portion passageway 22, and with tubular member passageway 16, as best shown in FIGS. 2 and 3.

Tip portion 30 further includes a second passageway 34 that extends angularly from, and communicates with, first passageway 32. Second passageway 34 includes a proximal opening 35 for receiving tension member 14, as further described herein. A generally annular ring 36 extends in the proximal direction along tip portion 30. Ring 36 is sized and shaped such that a proximal portion 37 of ring 36 is snugly received in main body portion channel 28. A more distal portion 38 of ring 36 receives an opening in locking arm 50, as described herein (FIG. 2). Tip portion 30 also includes a distal channel 40 for receiving the proximal end 19 of elongated tubular member 13. In a preferred embodiment, tip portion 30 is insert molded over tubular member proximal portion 19. Those skilled in the art will appreciate that tip portion 30 and elongated tubular member 13 may alternatively be engaged by other means well known in the art.

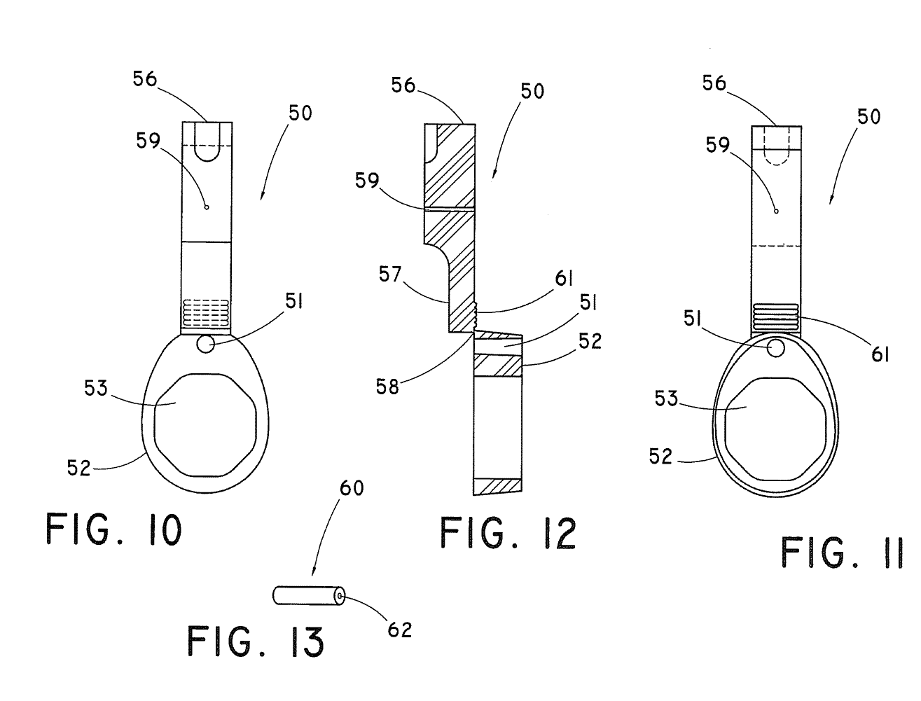

Locking arm 50 is further illustrated in FIGS. 10-12. FIG. 10 illustrates the locking arm when viewed from its orientation at the proximal end of the locking mechanism. FIG. 11 illustrates the locking arm when viewed from its orientation at the distal end, and FIG. 12 illustrates a side sectional view of the locking arm of the locking mechanism. As shown in FIGS. 10-12, locking arm 50 includes a generally annular or oval-shaped body portion 52, having an aperture 53 therethrough. If desired, aperture 53 may be shaped to facilitate receipt and positioning of body portion 52 over a complementary-shaped distal ring portion 38 of tip portion 30, as shown, e.g., in FIG. 2. In the non-limiting embodiment shown in FIGS. 10 and 11, aperture 53 has the shape of an octagon.

Locking arm 50 also includes a second aperture 51. In the preferred embodiment shown, second aperture 51 is disposed along the perimeter of body portion 52. Aperture 51 is sized and shaped to snugly receive a plug, or seal, 60 (FIG. 13).

Plug 60 is shown positioned in aperture 51 in FIGS. 2 and 3. The outer surface of plug 60 is preferably shaped (e.g., cylindrically-shaped) to enable a snug fit in a complementary-shaped aperture 51. Plug 60 is preferably formed of an elastomeric material (e.g., silicone, or an elastomeric block polymer or copolymer, such as KRATON), through which tension member 14 may be passed. Plug 60 is capable of sufficient flexure to allow easy passage of tension member 14 through passageway 34 upon activation of the locking mechanism (to be described). At the same time, plug 60 is sized and shaped to essentially fill, or “crowd”, passageway 34, thereby inhibiting leakage of fluids through the passageway. Preferably, plug 60 includes a passageway 62 suitable for passage of the tension member 14 therethrough.

Locking arm 50 also includes pivotable member 56. Pivotable member 56 is pivotally engaged with a surface of body portion 52, preferably via a living hinge 58. Pivotable member 56 is sized and shaped to be received in cradle 24 of main body portion 20 when the locking mechanism is maneuvered into the locked position. In the preferred embodiment shown, pivotable member 56 includes a contoured surface 57 having a shape complementary to that of ledge 23 of main body portion 20. The respective complementary surfaces meet in a manner such that a generally smooth outer surface of the locking mechanism is defined when the pivotable member 56 is pivoted to the closed position, as shown, e.g., in FIGS. 1 and 2. Pivotable member 56 includes an opening 59 extending therethrough for receiving tension member 14. Pivotable member 56 may be provided with knurls 61 to enhance control by the user, and to facilitate wrapping of the suture around the pivotable member. Wrapping a portion of the suture around the pivotable member provides an additional suture length to facilitate any possible repositioning of the shaped end of the tubular member that may be desired.

When the device is fully assembled and in the open, or unlocked, position shown in FIG. 3, tension member 14 follows the following path. This path may be most easily visualized when viewing the sectional views depicted in FIGS. 2 and 3. Tension member grasping end 45 extends radially outwardly from the catheter in a manner that allows convenient grasping and pulling by the operator. Tension member 14 extends from grasping end 45 through opening 59 in pivotable member 56, and if desired, may be wrapped one or more times around the length of pivotable member 56. Tension member 14 further extends through elastomeric plug 60 positioned in aperture 51 at a junction of locking arm 50 and tip member 30, and thereafter into tip member second passageway 34. Tension member 14 extends into elongated tubular member passageway 16, and extends therefrom substantially to the distal end of the tubular member. Tension member 14 thereupon passes out of the tubular member through one of ports 17, 18 (port 17 in FIG. 3) and then returns to the interior of the tubular member through the other port (port 18 in FIG. 3).

Ports 17, 18 may be positioned at various circumferential locations at the distal end of tubular member 13. In a preferred embodiment, they are spaced about 180 degrees apart along the circumference, as shown in FIG. 3. Tension member (e.g., suture) 14 then extends back in a proximal direction within tubular member passageway 16, whereupon end 46 of the tension member is fixedly captured at a juncture of main body portion 20 and tip member 30. This juncture is preferably disposed at an opposite radial side of the catheter 10 from the pivotable member 56. As stated, any excess length of the tension member end 46 that extends outside of the catheter through this juncture will typically be trimmed away after proper positioning of the catheter.

One method of using the drainage catheter with the locking mechanism will now be described. Initially, the distal end of the catheter is percutaneously inserted into the body cavity, such as the bladder. This step is typically performed by inserting the distal end of a thin-wall hollow needle through the abdominal wall and into the bladder in well-known manner. A wire guide is then inserted through the needle into the bladder, and the needle is removed, leaving the wire guide in place. A dilator may be used over the wire guide to increase the size of the puncture site.

During percutaneous insertion of catheter distal end 11 over the wire guide, the catheter will typically be manipulated into a generally straight configuration, with the locking mechanism in the unlocked position as shown, e.g., in FIG. 3. If desired, this generally straight configuration may be achieved and maintained by inserting a flexible stiffener (not shown) through the aligned passageways 22, 32, 16 (FIGS. 2 and 3) of the main body portion 20, tip portion 30 and elongated tubular member 13, respectively. Following insertion of the straightened distal end 11 of tubular member 13 into the bladder, the wire guide and flexible stiffener are removed from the patient. The distal end of the catheter is left in place for providing fluid flow from the bladder through ports 31 and/or distal tip 29 to a conventional fluid collection system (not shown), such as a proximally extending piece of tubing extending from connector 26 into a plastic collection bag, in well-known fashion.

In order to inhibit unintended withdrawal or dislodgement of the distal end of the catheter from the bladder, the locking mechanism 15 is manipulated to achieve the desired distal end configuration, such as the loop 16 shown in FIG. 1. To achieve this configuration, the operator grasps and pulls on end 45 of tension member 14, while the locking mechanism 15 is in the unlocked, or open, position (FIG. 3). As tension member grasping end 45 is pulled, tension member 14 is moved or drawn proximally to form distal end 11 into the desired loop configuration 12. In order to maintain distal end 11 in the desired loop configuration, the pivotable member 56 is pivoted in the proximal direction, such that it is received in cradle 24 of main body portion 20. With particular reference to the figures, it may be observed that pivotable member 56 pivots along living hinge 58, from the position shown in FIG. 3, to the position shown in FIG. 2. When the pivotable member is positioned in the locked configuration, tension member 14 is captured, or “locked”, between pivotable member 56 and cradle 24, as shown, e.g., in FIGS. 1 and 2. As a result, further pulling or other movement of the end 45 of tension member 14 is substantially prevented, thereby maintaining the tension that forms loop 12.

Those skilled in the art will recognize that not every feature of catheter 10 will be required in every instance, nor will every operating step described be required in every instance of use. Routine modifications may be made to the structure and the method of use from time to time, all of the foregoing being considered within the scope of the invention. Further, those skilled in the art will appreciate that although the locking mechanism 15 has been primarily described for use in connection with catheter 10, that locking mechanism 15 may also be used in combination with the tension member for locking other medical devices into a desired configuration.

Those skilled in the art will recognize that the various components of the catheter and locking mechanism described herein may be formed from a multiple of possible compositions that are suitable for such use. Typically, various polymeric compositions are desirable. For example, the main body portion 20 and tip portion 30 may be formed of polyurethane. In one embodiment, main body portion 20 may have a length of about 0.808 inch (20.5 mm), and a minimum internal diameter of about 0.136 inch (3.45 mm). Tip 30 may have a length of about 0.944 inch (24 mm) and a minimum internal diameter of about 0.136 inch (3.45 mm). The locking arm 50 may be formed from polypropylene. The various features of locking arm 50 are sized to correspond to the features of main body portion 20 and tip portion 30, as indicated in the figures. Typically, aperture 53 will have a major diameter of about 0.256 inch (6.5 mm).

Flexible tubular members suitable for use herein are well known in the art, and tubular member (catheter) 13 may be formed from any such well-known composition, such as polyether-urethane. Tubular member 13 will typically have, e.g., an outer diameter of between about 5 and 14 French, and a length of between about 15 and 40 cm. If desired, the distal portion, e.g., about the distal 5 cm, of the tubular member may have a hydrophilic coating. The suture may be formed of any compositions routinely used for sutures in the medical arts, such as monofilament nylon. As stated, plug 60 is typically formed of an elastomer (e.g., KRATON), and has a diameter, for example, of about 0.031 inch (0.79 mm).

It is intended that the foregoing detailed description be regarded as illustrative rather than limiting, and that it be understood that it is the following claims, including all equivalents, that are intended to define the spirit and scope of this invention.

1. A catheter comprising:
an elongated tubular member, said tubular member having a distal end for insertion into a patient, a proximal end, and a passageway extending longitudinally therethrough, said distal end being maneuverable into a desired configuration upon insertion, said distal end having a plurality of ports extending therethrough;
a locking mechanism engaged with said proximal end of said tubular member, said locking mechanism comprising a main body portion, a tip portion, and a locking arm;
said main body portion having a proximal end, a distal end, an outer surface, and a passageway extending therethrough, said outer surface sized and shaped such that a cradle portion is defined therealong;
said tip portion having a proximal end, a distal end, and a first passageway extending longitudinally therethrough, said tip portion further having a second passageway extending between a surface of said tip portion and said first passageway; said distal end of said tip portion engaged with said proximal end of said tubular member, and said proximal end of said tip portion engaged with said distal end of said main body portion, such that said main body portion passageway, said tip portion first passageway, and said tubular member passageway are aligned for fluid flow therethrough;
a locking arm, said locking arm including a body portion having first and second apertures extending therethrough, said first aperture received over a surface of one of said main body portion and tip portion, said second aperture communicating with said second passageway, said locking arm further including a pivotable member pivotally engaged with said body portion, said pivotable member having an opening extending therethrough, a portion of said pivotable member sized and shaped to be selectively received in and removed from said cradle portion;
an elastomeric sealing member, said elastomeric sealing member received in said second aperture and being sized and shaped for substantially sealing said second passageway from fluid flow therethrough when said tip portion first passageway and said tubular member passageway are in said alignment; and
a tension member extending along said elongated tubular member and said locking mechanism for selectively maneuvering said tubular member distal end into said desired configuration, an end of said tension member being receivable in said cradle portion when said elongated tubular member is in said desired configuration, and retainable therein upon receipt of said pivotable member portion in said cradle portion for locking said distal end in said configuration.

2. The catheter of claim 1, wherein said tension member includes a grasping end and a fixed end, said grasping end extending proximal of said locking arm for said maneuvering of said tubular member distal end; said tension member extending through said locking arm opening, said elastomeric member, said tip portion second passageway, said tip portion first passageway, and through said tubular member passageway to one of said ports at said tubular member distal end, and thereupon through a second port and extending in a proximal direction to an interior of said locking mechanism; said fixed end engaged with said locking mechanism, said grasping end retained in said cradle upon receipt therein of said pivotable member portion.

3. The catheter of claim 2, wherein said fixed end is engaged at an interior of said locking mechanism at a juncture of said main body portion and said tip portion.

4. The catheter of claim 2, wherein said tension member is wrapped around said pivotable member of said locking arm one or more times.

5. The catheter of claim 2, wherein said tension member extends interiorly through said elongated tubular member and passes through a first one of said ports such that the tension member extends a distance exterior of said tubular member, said tension member passing through a second port to said tubular member interior, and wherein said tubular member distal end includes one or more drainage ports spaced therealong.

6. The catheter of claim 5, wherein said first port and said second port are spaced about 180 degrees along the circumference of the tubular member.

7. The catheter of claim 5, wherein said tubular member has an open distal tip.

8. The catheter of claim 1, wherein said elastomeric sealing member includes an opening therethrough for passage of said tension member.

9. The catheter of claim 1, wherein the cradle portion includes a surface complementary to a surface of the locking arm pivotable member, such that upon receipt of the pivotable member in the cradle portion a substantially smooth locking mechanism outer surface is defined thereby.

10. A locking mechanism for use with a tension member to lock a medical device into a desired configuration, comprising:
a main body portion having an outer surface, and having a passageway extending therethrough, said outer surface having a contoured surface therealong; a tip portion having a first passageway extending longitudinally therethrough, and a second passageway extending between a surface of said tip portion and said first passageway; said locking mechanism structured such that main body portion passageway and said tip portion first passageway are aligned for permitting fluid flow therethrough; and
a locking arm, said locking arm including a body portion having a first aperture extending therethrough, said first aperture received over a surface of one of said main body portion and tip portion, said locking arm further having a second aperture, said second aperture communicating with said second passageway, said locking arm including a pivotable member having a surface, said pivotable member movable between a first position whereupon a tension member is movable along said locking mechanism to draw said medical device into a desired configuration, and a second position whereupon said pivotable member surface engages said main body portion contoured surface, such that said tension member is lockingly positioned between said pivotable member and main body portion and said medical device is locked into said configuration.

11. The locking mechanism of claim 10, wherein said main body portion contoured surface and said pivotable member surface are structured and arranged such that upon engagement of said surfaces a generally smooth outer surface of said locking mechanism is defined thereby.

12. The locking mechanism of claim 11, wherein said main body portion contoured surface comprises a cradle, said cradle including a ledge disposed therealong.

13. The locking mechanism of claim 10, further comprising an elastomeric seal member received in said second aperture, said elastomeric seal member sized and shaped for sealing said second passageway from fluid flow therethrough.

14. A method for percutaneously draining fluid from a cavity of a patient, comprising:
providing a drainage catheter comprising a locking mechanism and an elongated tubular member extending distally from the locking mechanism, the elongated tubular member having a distal end for insertion into a patient, a proximal end, and a passageway extending longitudinally therethrough, said distal end being maneuverable into a desired configuration upon insertion, said distal end having a plurality of ports extending therethrough; the locking mechanism comprising a main body portion, a tip portion, and a locking arm; the main body portion having a proximal end, a distal end, an outer surface, and a passageway extending therethrough, said outer surface sized and shaped such that a cradle portion is defined therealong; the tip portion having a proximal end, a distal end, and a first passageway extending longitudinally therethrough, said tip portion further having a second passageway extending between a surface of said tip portion and said first passageway; said distal end of said tip portion engaged with said proximal end of said tubular member, and said proximal end of said tip portion engaged with said distal end of said main body portion, such that said main body portion passageway, said tip portion first passageway, and said tubular member passageway are aligned for fluid flow therethrough; a locking arm, the locking arm including a body portion having first and second apertures extending therethrough, said first aperture received over a surface of one of said main body portion and tip portion, said second aperture communicating with said second passageway, said locking arm further including a pivotable member pivotally engaged with said body portion, said pivotable member having an opening extending therethrough, a portion of said pivotable member sized and shaped to be selectively received in and removed from said cradle portion; an elastomeric sealing member, the elastomeric sealing member received in said second aperture and being sized and shaped for substantially sealing said second passageway from fluid flow therethrough when said tip portion first passageway and said tubular member passageway are in said alignment; and a tension member extending along said elongated tubular member and said locking mechanism for selectively maneuvering said tubular member distal end into said desired configuration;
percutaneously inserting the distal member end of the elongated tubular member into the cavity of a patient;
grasping an end of said tension member, and moving the tension member to maneuver the elongated tubular member distal end into the desired configuration, in a manner such that a portion of the tension member is received in said cradle portion; and
pivoting said locking arm pivotable member such that said pivotable member is received in said cradle and said tension member portion is captured between said pivotable member and said cradle, whereby the tubular member distal end is locked into the desired configuration.

15. The method of claim 14, wherein the tension member is wrapped around the pivotable member of the locking arm one or more times.

16. The method of claim 14, wherein the tension member extends interiorly through said elongated tubular member and passes through a first one of said ports such that the tension member extends a distance exterior of said tubular member, said tension member passing through a second port to said tubular member interior, and wherein said tubular member distal end includes one or more drainage ports spaced therealong.

17. The method of claim 16, wherein said first port and said second port are spaced about 180 degrees along the circumference of the tubular member, and said tubular member has an open distal tip.

18. The method of claim 14, wherein said elastomeric sealing member includes an opening therethrough for passage of said tension member.

19. The method of claim 14, wherein the cradle portion includes a surface complementary to a surface of the locking arm pivotable member, such that upon receipt of the pivotable member in the cradle portion a substantially smooth locking mechanism outer surface is defined thereby.

20. The method of claim 14, wherein the tension member comprises a suture.
